SEQUENCE LISTING (5 sequences)

SEQ ID NO: 1 - protein, 350 aa, Unknown
  DNA isolated from environmental samples containing uncultured microorganism
MQNLPAGTKGFDFLEFTTTKPRVLEQQFLSMGFVPVAKHNSIDITIYQQNDIRFIINADKNTLAHQFAQHGPSVSAMGFLVESAEDAYQYAITHGAQSYRPTQEVVPYTMPAIQGVGGSLIYFVDYKQGKANYLQHFSVLPTASAVKNAGLTYIDHVTHNLHRGNMNQWAEFYENIFNFKEVRYFDIEGRLTGLKSRAMTSPCGKIRIPLNESSDDKSQIEEFLKDFNGEGIQHIALGTNDIYESVAQLHTKGLEFLDTPETYYDLIEKRLPKHGESVERLRKNRILIDGTTETERKILLQIFTKNMLGPVFFEIIQRKGDEGFGEGFKALFESIELDQIKRGVLQVEQS

SEQ ID NO: 2 - protein, 348 aa, Legionella pneumophila
MQNNNPCGLDGFAFLEFSGPDRNKLHQQFSEMGFQAVAHHKNQDITLFKQGEIQFIVNAASHCQAEAHASTHGPGACAMGFKVKDAKAAFQHAIAHGGIAFQDAPHANHGLPAIQAIGGSVIYFVDEEHQPFSHEWNITSPEPVVGNGLTAIDHLTHNVYRGNMDKWASFYASIFNFQEIRFFNIKGKMTGLVSRALGSPCGKIKIPLNESKDDLSQIEEFLHEYHGEGIQHIALNTNDIYKTVNGLRKQGVKFLDVPDTYYEMINDRLPWHKEPLNQLHAEKILIDGEADPKDGLLLQIFTENIFGPVFFEIIQRKGNQGFGEGNFQALFEAIERDQVRRGTLKELS

SEQ ID NO: 3 - protein, 356 aa, Vibrio vulnificus
MVDAINPLGTDGFEFVEYTAADNTGIEQLKHLFSSLGFAEVAKHRSKEAWLYRQGDINFVVNAQPHSQEEFAKVHGPSVCGMAFRVQDAASALKHALTNGAEEYKTEIGPMELSIPAVYGIGGSLLYFVDRYGKQSIYDVDFRFYDDAAQRLAKSDVGLYEIDHLTHTLKRRNMDTWAGSMSVLVISVKIRYFDIERKLTGLVSRAMNAPCGKIRIPINESSDDKSQIEEFIREYNGEGIQHIALTTDDIYQTVQTLRDRGMDFMPTPDTYYDKVDSRVEGHKENVSRLRDLRILIDGAPLKDGILLQIFTQTVIGPVFFEIIQRKGNEGFGEGNFKALFESIEEDQIRRGVLNNA

SEQ ID NO: 4 - protein, 357 aa, Pseudomonas sp.
ADLYENPMGLMGFEFIELASPTPNTLEPIFEIMGFTKVATHRSKDVHLYRQGAINLILNNEPHSVASYFAAEHGPSVCGMAFRVKDSQKAYKRALELGAQPIHIETGPMELNLPAIKGIGGAPLYLIDRFGEGSSIYDIDFVFLEGVDRHPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLSDDLIKTWDHLKSIGMRFNTAPPDTYYEMLEGRLPNHGEPVGELQARGILLDGSSESGDKRLLLQIFSETLMGPVFFEFIQRKGDDGFGEGNFKALFESIERDQVRRGVLSTD

SEQ ID NO: 5 - protein, 346 aa, Shewanella colwelliana
MASEQNPLGLLGIEFTEFATPDLDFMHKVFIDFGFSKLKKHKQKDIVYYKQNDINFLLNNEKQGFSAQFAKTHGPAISSMGWRVEDANFAFEGAVARGAKPAADEVKDLPYPAIYGIGDSLIYFIDTFGDDNNIYTSDFEALDEPIITQEKGFIEVDHLTNNVHKGTMEYWSNFYKDIFGFTEVRYFDIKGSQTALISYALRSPDGSFCIPINEGKGDDRNQIDEYLKEYDGPGVQHLAFRSRDIVASLDAMEGSSIQTLDIIPEYYDTIFEKLPQVTEDRDRIKHHQILVDGDEDGYLLQIFTKNLFGPIFIEIIQRKNNLGFGEGNFKALFESIERDQVRRGVL

(19) United States
(12) Patent Application Publication
(10) Pub. No.: US 2001/0047029 A1
(43) Pub. Date: Nov. 29, 2001

(54) Triaryl cation antibiotics from environmental DNA

(76) Inventors: Jo E. Handelsman, Madison, WI (US); Robert M. Goodman, Madison, WI (US); Doreen E. Gillespie, Madison, WI (US); Alan D. Bettermann, Madison, WI (US); Jon C. Clardy, Ithaca, NY (US); Sean F. Brady, Ithaca, NY (US)

Correspondence Address: Intellectual Property Department, DEWITT ROSS & STEVENS, S.C., Firstar Financial Centre, 8000 Excelsior Drive, Suite 401, Madison, WI, 53717-1914, US

(21) Appl. No.: 09/791,961

(22) Filed: Feb. 23, 2001

Related U.S. Application Data
(63) Continuation-in-part of application No. 09/558,712, filed on Apr. 26, 2000.

(51) Int. Cl. C07D 209/02; C07D 405/00; C07D 409/00; C07D 411/00

(52) U.S. Cl. 514/415; 435/7.1; 548/465

(57) ABSTRACT

Disclosed are triaryl cationic compounds that exhibit broad spectrum antibiotic and antifungal activity, pharmaceutical compositions containing the compounds, and methods of treating bacterial and fungal infections using the compounds. The compounds were initially isolated by screening a 25,000-member bacterial artificial chromosome (BAC) library of environmental (eDNA) from soil. At least one clone produced a dark brown melanin-like compound that was found to have antibiotic activity. The compounds were isolated and synthesized de novo. From within the positive clone, a single open reading frame that shares extensive sequence similarity with members of the 4-hydroxyphenylpyruvate family of enzymes was found to be necessary and sufficient to confer the production of at least one of the subject compounds on E. coli.

### CROSS-REFERENCE TO RELATED APPLICATIONS

This is a Continuation-In-Part of co-pending application Ser. No. 09/558,712, filed Apr. 26, 2000, the entirety of which is incorporated herein by reference.

### FIELD OF THE INVENTION

The invention relates to antibiotics derived from microorganisms. In particular, the invention relates to antibiotic compounds having a triaryl-substituted methane core, pharmaceutical compositions containing the same, and methods of making the same.

### BIBLIOGRAPHIC CITATIONS

Complete bibliographic citations to the references discussed herein are contained in the Bibliography section, directly preceding the claims.

### BACKGROUND OF THE INVENTION

Cultured microorganisms produce an extraordinary array of structurally diverse and useful organic compounds. Molecular diversity measurements of microbial communities show that cultured microflora represent only a small fraction of the microbial diversity present in any natural environment. Currently unculturable microorganisms undoubtedly produce potentially valuable compounds and general methods to access these compounds are needed. An approach to study the natural products produced by uncultured microorganisms by introducing large fragments of DNA extracted directly from environmental samples (eDNA) into E. coli is described in co-pending parent application Ser. No. 09/558,712, filed Apr. 26, 2000. Heterologous expression of eDNA in an easily cultured host will provide access to the genomes of many uncultured microorganisms while bypassing the need to culture these organisms.

Natural products have traditionally been a rich source of small organic compounds that have found multiple uses in our daily lives. From the earliest known dyes and intoxicants to the most recent anti-neoplastic agents, natural products have been used by all human societies. The large collection of organic compounds that have been characterized from microbial sources is now known to have arisen from only a small sample of the earth's microbial diversity. Nucleic acids-based studies suggest that only a tiny fraction of any natural microbial community is cultured using standard microbiological methods. Uncultured bacteria have now been identified in a number of different environments including marine (1), gut (2, 3), hot springs (4), and soil (5, 6, 7). Uncultured microorganisms, therefore, represent one of the largest remaining unstudied pools of biodiversity. However, because these organisms are resistant to culturing, the natural products they produce remain inaccessible.

Using cloning and heterologous expression of large fragments of microbial DNA extracted directly from environmental samples (environmental DNA, eDNA) provides a means to access this previously untapped biodiversity. This approach has been termed “cloning the metagenome” to refer to accessing the collective genomes of the organisms in an environment (8). With the advent of bacterial cloning vectors such as the bacterial artificial chromosome (BAC), a vector that faithfully replicates very large fragments of DNA (9), it is now possible to clone and express in easily cultured hosts eDNA that codes for natural products produced by uncultured microorganisms. In short, direct cloning of eDNA provides a means to access the biosynthetic capacity of the genomes of heretofor uncultured microorganisms.

A metagenomics approach was first used to study the uncultured microflora present in soil samples. As with all other natural environments studied to date, the diversity of uncultured microorganisms in soil far exceeds that of cultured microorganisms. DNA-DNA reassociation kinetics and light microscopy analysis of soil samples indicate that cultured microorganisms represent less than 1% of the actual microbial diversity that exists in soil (10, 11). Analyzing small subunit rRNA gene sequences cloned from PCR-amplified soil DNA indicates that the uncultured microbial diversity in soil is much larger and more diverse than previously believed (5, 6, 7, 12). In addition to known examples of Archaea, Bacteria and Eukarya, analysis of small subunit rRNA gene sequences from soil microflora has consistently identified many sequences from previously uncharacterized microbial taxa. A recent analysis showed that nearly one third of the major divisions of phyla within the domain Bacteria are known only as environmental rRNA sequences (containing no cultivated members) and many of the well known divisions contain a wide diversity of as-yet- uncultivated members (13). Although there appears to be no easy way to culture this large collection of unstudied microorganisms, it is possible to isolate large fragments of microbial DNA directly from soil samples in high yields (>1 μg of DNA/gram of soil) (14, 15). The molecular diversity data from soil, together with the ease with which large eDNA can be harvested from soil samples, makes soil an ideal candidate for this new approach to the discovery of natural products.

Described herein are triaryl cationic antibiotics that are produced at elevated levels by a member of this library, pharmaceutical compositions containing the antibiotics, and methods of treating microbial infection using the pharmaceutical composition. The triaryl antibioutics were initially generated using a 25,000-member BAC library of eDNA extracted from soil (15; co-pending and co-owned U.S. patent application Ser. No. 09/558,712, filed Apr. 26, 2000), and subsequently synthesized de novo.

### SUMMARY OF THE INVENTION

A first embodiment of the invention is directed to a pharmaceutical composition for treating microbial or fungal infections. The composition comprising an antibiotic-effective or an antifungal-effective amount of a compound having a structure as shown in Formula I or Formula II:

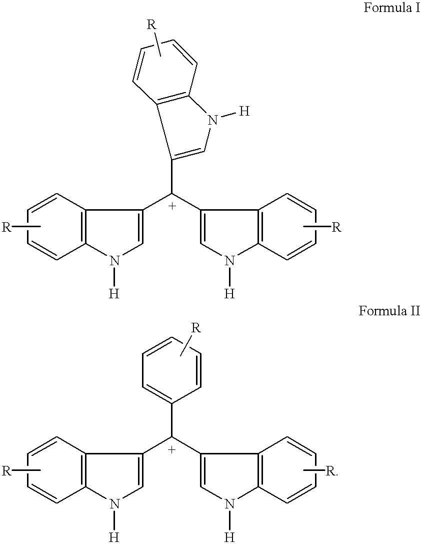

Each R group in the formulae is independently selected from the group consisting of hydrogen, halo, hydroxy, amino, unsubstituted C₁ to C₆-alkyl, and C₁ to C₆-alkyl substituted with one or more of halo, hydroxy, or amino. Expressly included within the scope of the invention are all stereo-isomers (i.e., individual isolated and/or enriched enantiomers or diasteromers, and racemic mixtures thereof) and pharmaceutically-acceptable salts thereof. The composition optionally is formulated in combination with a pharmaceutically-acceptable carrier. In the preferred embodiment, each R substituent is hydrogen or unsubstituted alkyl. The Formula I compound wherein each R is hydrogen has been given the trivial name turbomycin A; the Formula II compound wherein each R is hydrogen has been given the trivial name turbomycin B.

The composition displays antibiotic and antifungal activity and hence finds utility as a composition for the treatment of microbial and fungal infections. Thus, a second embodiment of the invention is drawn to treating bacterial or fungal infection in a subject in need thereof, the method comprising administering to the subject an antibiotic- or antifungal-effective amount of a compound of Formula I or Formula II. Such treatment expressly includes treatment of such infections in mammals, including humans.

A third embodiment of the invention is directed to novel compounds that exhibit antibiotic and antifungal activity. The compounds selected from the group consisting of:

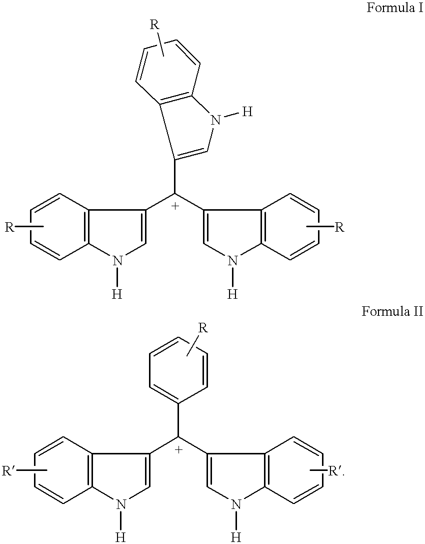

Here, each R substituent in the Formula I structure is independently selected from the group consisting of hydrogen, halo, hydroxy, amino, unsubstituted C₁ to C₆-alkyl, and C₁ to C₆-alkyl substituted with one or more of halo, hydroxy, or amino, provided that each R group is not simultaneously hydrogen.

Each R′ in the Formula II structure is independently selected from the group consisting of hydrogen, halo, hydroxy, amino, unsubstituted C₁ to C₆-alkyl, and C₁ to C₆-alkyl substituted with one or more of halo, hydroxy, or amino.

Expressly included within the scope of the recited compounds are all stereo-isomers (i.e., individual isolated and/or enriched enantiomers or diasteromers, and racemic mixtures thereof) and salts thereof. The preferred compound of Formula I has two R groups that are hydrogen, with the third R group being alkyl; the preferred compound of Formula II has all three R′ groups being hydrogen.

A fourth embodiment of the invention is drawn to an isolated polypeptide that, when present in a microbe, results in the production by the microbe of compounds shown in Formulae I and II wherein each R (and/or R′) is hydrogen. The isolated polypeptide is shown in SEQ. ID. NO. 1. The invention is also directed to an isolated polynucleotide that encodes the polypeptide as shown in SEQ. ID. NO. 1, and which drives the production of compounds of Formulae I and II wherein each R (and/or R′) are hydrogen in microbes transformed to contain and express the polynucleotide.

Also described herein is the successful heterologous expression of eDNA extracted directly from soil as a means to access previously uncharacterized small organic compounds.

Further disclosed herein are methods for making the compounds of Formulae I and II. For example, to make a the compound of Formula I wherein all of the R substituents are hydrogen, a mixture of indole-3-carboxyaldehyde, indole, acetic acid, and ethanol is heated and neutralized to form a tri-substituted methane, which is subsequently oxidized. The oxidized substituted methane is then gently heated and the pH is increased. The resulting product is extracted to yield a crude reaction product containing the desired end product. The crude reaction product may then be further purified, if desired, by an number of means, such as by chromatography or recrystallization from an anti-solvent.

Compounds of Formula II can be made by an analogous method. For example, the compound of Formula II wherein each R substituent is hydrogen is made by heating a mixture of benzaldehyde, indole, acetic acid, and ethanol. The composition is neutralized to form a tri-substituted methane, which is then oxidized. The oxidized substituted methane is then heated and the pH is increased. The product can then be extracted an purified as noted herein.

A primary advantage of the invention is that it provides compounds and compositions effective to treat bacterial and fungal infections, including bacterial and fungal infections in humans. The infections that can be treated using the subjection compounds and compositions include, without limitation: Erwinia herbicola, Escherichia coli, Salmonella typhimurium, Bacillus cereus, Bacillus subtilis, Staphylococcus aureus, Streptococcus pyogenes, Streptomyces griseus, and Candida guilliermondii.

Another advantage of the present invention is that it provides a means to discover new compounds by direct cloning of eDNA taken from uncultured organisms. In short, the approach described herein demonstrates the feasibility and desirability of using direct cloning of eDNA to discover new compounds, including both new genes and their encoded polypeptides, and well as novel small organic molecules such as the novel antibiotic compounds described herein.

Further advantages, features, and objects of the invention will be apparent from the following detailed description of the invention in conjunction with the associated drawings.

### BRIEF DESCRIPTION OF THE DRAWINGS

FIG. 1 illustrates ORFs and GPS transposon insertion sites in the eDNA subclone G4P8. Shaded arrows indicate transposon insertions that did not disrupt turbomycin A and B accumulation; unshaded arrows indicate insertions that resulted in the lack of turbomycin A and B accumulation.

FIG. 2 depicts an alignment of ORFI with Lly (L. pneumophila) (ATCC No. Q53407), V11Y (V. vulnificus) (ATCC No. 006695), 4HPPD (Pseudomonas sp.) (ATCC No. P80064), and MelA (S. colwelliana) (ATCC No. P23996). Identical base pairs are underlined.

### DETAILED DESCRIPTION OF THE INVENTION

Initial Identifcation and Isolation of Compounds:

A 25,000-member bacterial artificial chromosome (BAC) library of eDNA from soil was screened for the production of colored compounds, and a clone, P57-G4, that produced a dark brown melanin-like color was identified. The methanol extract of the acid precipitate from the culture broth of P57-G4 contained elevated levels of two triaryl, cationic compounds that were given the trivial names turbomycin A and B. (Turbomycin A is the compound of Formula I wherein all R groups are hydrogen; turbomycin B is the compound of Formula II wherein all R groups are hydrogen.) Both of turbomycin A and B were found to exhibit broad spectrum antibiotic activity. A single open reading frame within P57-G4 that shares extensive sequence similarity with members of the 4-hydroxyphenylpyruvate family of enzymes was necessary and sufficient to confer the production of color on E. coli. (For further discussion, see the Examples.)

¹³C NMR spectra were recorded on a Varian Unity 400 spectrometer. ¹H and two-dimensional spectra were recorded on a Varian Unity 500 spectrometer. ¹H-¹³C HMQC and H-¹³C HMBC experiments were run using pulse field gradients. Mass spectral data were acquired.

To isolate the natural products from the culture broth, cell-free culture broth from P57-G4 grown in LB medium for 48 hours (37° C.) was brought to pH 13 with NaOH, stirred for one hour, and then acidified to pH 2 with HCl. Separate methanol extracts of the precipitate wer collected by filtration after 24 hours and 72 hours. Each extract as then resuspended in 80:20 chloroform:methanol and the chloroform:methanol solution was then passed over a silica plug to remove any remaining insoluble material.

The eluent from the silica plug was partitioned by silica gel chromatography using a chloroform:methanol step gradient modified with 0.2% acetic acid (95:5, 90:10, 80:20, and 0:100). The 90:10 fraction was dried, reapplied to a silica column and developed using a chloroform:methanol step gradient modified with 0.1% triethylamine (95:5, 90:10, 80:20, and 0:100). Fractions from both the 24 and 72 hour precipitates containing turbomycin A were pooled from the 80 to 90% chloroform eluent, while fractions containing turbomycin B were pooled from the 95% chloroform fluent. Turbomycin A (R_{f} 0.39, silica gel TLC, chloroform:methanol:acetic acid, 80:20:0.2) was obtained from a final normal phase column (85:15 chloroform:methanol modified with 0.2% acetic acid). Turbomycin B (R_{f}0.73, silica gel TLC, chloroform:methanol: acetic acid, 80:20:0.2) was obtained from a final normal phase column (step gradient, 93:7 and 86:14 modified with 0.2% acetic acid).

NMR and mass spectrometry data (see also Table 1):

Turbomycin A: HRMS-FAB (m/z): (M)+ calculated for C₂₅H₁₈N₃ 360.1501; found 360.1498; ¹H NMR (500 MHz, CD₃OD referenced at 3.31 ppm) 8.15 (3H, s, H1, 1′, 1″), 7.66 (3H, d, 10.5, H7, 7′, 7″), 7.35 (3H, t, d, 8.5, 2, H6, 6′, 6″), 7.08 (3H, t, 9, H5, 5′, 5″), 7.04 (3H, bd, 10, H4, 4′, 4″); ¹³C NMR (100 MHz, CD₃OD referenced at 49.15 ppm) 143.1 (C2, 2′, 2″), 121.3, (C3, 3′, 3″), 128.3 (C3 a, 3a′, 3a″), 122.5 (C4, 4′,4″), 124.7 (C5, 5′,5″), 126.3 (C6, 6′,6″), 114.7 (C7, 7′, 7″), 140.1 (C7a, 7a′, 7a″), 163.1 (C8).

Turbomycin B: HRMS-FAB (m/z): (M)+ calculated for C23H₁₇N₂ 321.1392 found 321.1389; ¹HNMR (500 MHz, CD₃OD referenced at 3.31 ppm) 8.24 (2H, s, H2, 2′), 7.87 (m, H12), 7.68 (6H, m, H7, 7′, 10, 14, 11, 13), 7.41 (2H, t, 7, H6, 6′), 7.25 (2H, t, 7, H5, 5′), 6.87 (2H, d, 8, H4, 4′); ¹³C NMR (100 MHz, CD₃OD referenced at 49.15 ppm) 147.8 (C2, 2′), 124.0 (C3, 3′), 128.3 (C3a, 3a′), 123.1 (C4, 4′), 125.8 (C5, 5′), 127.5 (C6, 6′), 115.5 (C7, 7′), 141.8 (C7a, 7a′), 171.4 (C8), 140.4 (C9), 130.6* (C10, 14), 134.7* (C 1, 13), 135.0 (C 12) *NMR assignment are ambiguous.

TABLE 1
¹H and ¹³C NMR data for Turbomycin A and B

| Turbo- | | | Turbo- | | |
|---|---|---|---|---|---|
| mycin | | | mycin | | |
| B | ¹³C^{a} | ¹H^{b,c} | A | ¹³C^{a} | ¹H^{b,c} |
| 1, 1′, 1″ | | NH | 1, 1′ | | NH |
| 2, 2′, 2″ | 143.1 | 8.15 (3H, s) | 2, 2′ | 147.8 | 8.24 (2H, s) |
| 3, 3′, 3″ | 121.3 | 3, 3′ | 124.0 | | |
| 3a, 3a′, | 128.3 | | 3a, 3a′ | 128.3 | |
| 3a″ | | | | | |
| 4, 4′, 4″ | 122.5 | 7.04 (3H, bd, 10) | 4, 4′ | 123.1 | 6.87 (2H, d, 8) |
| 5, 5′, 5″ | 124.7 | 7.08 (3H, t, 9) | 5, 5′ | 125.8 | 7.25 (2H, t, 7) |
| 6, 6′, 6″ | 126.3 | 7.35 (3H, t, d, 8.5, | 6, 6′ | 127.5 | 7.41 (2H, t, 7) |
| | | 2) | | | |
| 7, 7′, 7″ | 114.7 | 7.66 (3H, d, 10.5) | 7, 7′ | 115.5 | 7.68 (2H, m) |
| 7a, 7a′, | 140.1 | | 7a, 7a′ | 141.8 | |
| 7a″ | | | | | |
| 8 | 163.1 | | 8 | 171.4 | |
| | | | 9 | 140.4 | |
| | | | 10, 14^{d} | 130.6 | 7.68 (2H, m) |
| | | | 11, 13^{d} | 134.7 | 7.68 (2H, m) |
| | | | 12 | 135.0 | 7.87 (m) |

^{a}referenced at 49.15 ppm in CD₃OD
^{b}referenced at 3.31 ppm in CD₃OD
^{c}assignments determined by ¹H-¹³C HMQC
^{d}assignment uncertain

DeNovo Synthesis of Turbomycin A and B:

Turbomycin A and B were made by heating 250 μmoles of the appropriate aldehyde (indole-3-carboxyaldehyde for turbomycin A and benzaldehyde forturbomycin B), 500 μmoles indole, 40 μl acetic acid, and 360 μl ethanol at 80° C. for 4 hours. The reaction was neutralized with 10% NaOH, and the tri-substituted methane oxidized in situ by adding 200 mg tetrachloro-1,4-benzoquinone and heating at 80° C. for 40 minutes. An equal volume of 10% NaOH was then added and the reaction extracted 3 times with ethyl acetate to obtain a crude reaction product.

Synthetic turbomycin A, spectroscopically identical to the natural product, was obtained from the crude indole-3-carboxyaldehyde reaction product following two normal phase chromatography steps (90:10:0.1 chloroform:methanol:triethylamine followed by 80:20:0.1 chloroform:methanol: acetic acid).

Synthetic turbomycin B, spectroscopically identical to the natural product, was obtained from the crude benzaldehyde reaction product following two normal phase chromatography steps (98:2:0.1 chloroform:methanol:triethylamine followed by 90:10:0.1 chloroform:methanol:acetic acid).

Synthesis of the substituted forms of the Formula I and II compounds is accomplished in the same fashion as described above, using an appropriately substituted indole-carboxyaldehydes to yield corresponding compounds of Formula I wherein one or more R groups is a substituent other then hydrogen and using an appropriately substituted benzaldehydes to yield corresponding compounds of Formula II wherein one or more R groups is a substituent other then hydrogen.

X-ray Crystal Structure Determination of Synthetic Product:

Synthetic turbomycin A was converted to the perchlorate salt and then crystallized from acetone and water by slow evaporation. An orange block crystal (0.50×0.40×0.30 mm³) was mounted on a Bruker “SMART” diffractometer equipped with a 3KW sealed tube (MoKₐ) X-ray generator. The perchlorate salt of synthetic turbomycin B crystallized in the P1 bar space group with unit cell dimensions a=10.5517 (9) Å, b=10.8951 (9) Å, c=12.223 (1)Å. A hemisphere of data was taken using a narrow-frame method (1321 frames, 0.3° ω-scan, exposure time of 30 seconds/frame, 2θₘₐₓ=52.42°). Raw data were integrated with the Bruker “SAINT” program to yield a total of 6313 reflections, of which 4266 were independent (Rᵢₙₜ=2.94%), and 3200 were above 4σ(F). Data were corrected for adsorption using the “SABAB3S” program. The structure was solved by direct methods and refined by full matrix least squares on F² techniques using anisotropic displacement parameters for all non-hydrogen atoms and hydrogen atoms were included in all calculated positions. At final convergence, R₁=8.79% and GOF=1.307 for 407 parameters. Archival data have been deposited with the Cambridge Crystallographic Data Center.

Genetic Manipulation of eDNA Required for Turbomycin Production:

Many of the steps noted herein for the manipulation of DNA, including digesting with restriction endonucleases, amplifying by PCR, hybridizing, ligating, separating and isolating by gel electrophoresis, transforming cells with heterologous DNA, selecting successful transformants, and the like, are well known and widely practiced by those skilled in the art and are not extensively elaborated upon herein. Unless otherwise noted, the DNA protocols utilized herein are described extensively in Sambrook, J.; Fritsch, E. F.; Maniatis, T. (1989), Molecular Cloning: A Laboratory Manual; Cold Spring Harbor Laboratory Press: New York, N.Y.

E. coli DH10B was used for all cloning and culturing. The library was constructed in pBeloBAC11, as previously described (15).

P57-G4 was digested with PstI and shotgun cloned into pGEM-5Zf+ (Promega, Madison, Wis.) following standard techniques (26). The “GPS-1” Genome Priming System (NEB; Beverly, Mass.) was used to generate transposon insertions according to the supplier's instructions. Donor plasmid and G4P8 were mixed at a X:X molar ratio, and transformants were selected on kanamycin/chloramphenicol.

DNA sequencing was performed on an ABI Model 377 automated sequencer. Sequence similarity searches were conducted using BLAST (27), and other analyses were conducted with “DNASTAR” software (Madison, Wis.).

Construction of a Clone Expressing Legiolysin:

To determine whether the legiolysin (lly) gene of Legionella pneumophila conferred the same activity in E. coli as the putative ORF1 sequence in G4P8 cloned from eDNA (the sequence that confers production of turbomycin A and B (see Examples), lly-specific primers were designed based on sequences in GenBank and used to amplify the lly coding sequences and upstream promoter regions according to standard protocols (28). The PCR product was cloned into the vector pGEM-T (Promega; Madison, Wis.) and electroporated into DH10B. Clones containing the lly gene were verified by restriction digestion and homogentisate-melanin pigment production.

Cell-free Assay for Turbomycin Production:

The potential interaction of homogentisate (HGA) (an intermediate in the tyrosine degradation pathway) and indole compounds in the formation of turbomycins in a cell-free assay was investigated by the incubation of HGA and indole compounds in growth conditions identical to those employed for the E. coli clones. A range of HGA, indole, and indole-3-carboxaldehyde concentrations (1 μM to 1 mM) were added to 10 ml LB medium, separately and in combination, followed by incubation at 37° C. in aerated 1 8-mm culture tubes for 48 hours. The cell-free media were then adjusted to pH 13 with NaOH, stirred for 1 hour, and acidified to pH 2 with HCl for 48 hours at room temperature. The dark brown precipitate was collected by filtration and then extracted with methanol. The methanol extract was then resolved by TLC as described above.

Zone of Inhibition Assays:

Organisms were streaked onto Brain Heart Infusion plates containing 5% yeast extract. Escherichia coli (H5997), Staphylococcus aureus (3001), Enterococcus faecalis (4025), Streptococcus pyogenes (8P01), and Pseudomonas aeruginosa (9020) were incubated at 37° C.; Bacillus subtilis (BR151pPL608), Bacillus cereus var. mycoides (1003), Erwinia herbicola (IRQ), Salmonella typhimurium (LT2), Streptomyces griseus (6501), and Candida guilliermondii (Y001) were incubated at 28° C. A single colony from each organism was inoculated into 10 ml of BHI broth in a 100 ml flask. All cultures were shaken at 200 rpm overnight (12 hours).

Screening plates were prepared by adding 200 μl of the overnight cultures to 20 ml of cooled medium (BHI/YE). Natural and synthetic turbomycin A and turbomycin B, dissolved in DMSO to a concentration of 250 μg/ml, were applied as 10 μl drops to the surface of the plate. The plates were placed at 4° C. overnight (12 hours), then incubated at either 37° C. or 28° C. until a lawn developed. Inhibition was scored visually and zones of inhibition reported as the diameter of the zone in millimeters. The assay was conducted in duplicate plates.

Pythium ultimum (1033) and Fusarium solani f. sp. glycine (90.1) were grown on ¼- strength potato dextrose agar (29) at 23° C. Plugs were cut from the fungal lawn using a #5 cork borer and placed onto the center of a fresh plate containing the DMSO-dissolved compounds. The plates were incubated 7 days, or until the organism covered the plate, at 23° C., in the dark.

Minimum Inhibitory Concentration (MIC) Determination:

Overnight cultures were prepared as for the plate inhibition assays. Culture optical densities (O.D.) were normalized to 0.15 (150 μl path length) by performing 1:1 serial dilutions (150 μl transfer into 150 μl diluent) in a 96 well microtiter plate. Inoculum cell concentrations (colony forming units/ml) were subsequently determined by performing 1:5 serial dilution series (50 μ transfer into 200 μl diluent) in a second microtiter plate. Three dilutions were plated (3.13×10³, 1.56×10⁴, 7.81×10⁵) by mixing 50 μl of a given dilution into 20 ml of cooled BHI/YE medium and quickly pouring into a petri plate.

Test compounds, both natural and synthetic, were dissolved in DMSO (2. 0 mg/ml) and diluted into BHI/YE medium to a concentration of 106 μg/ml (5.35% v/v DMSO). Samples of each of the test compounds (150 μl each) were added to 3 separate wells, each containing 150 μl of BHI/YE medium (no DMSO). Serial dilutions (1:1) were performed (150 μl transfer into 150 μl diluent containing 2.6% DMSO) to achieve final concentrations of 50, 25, 12.5, 6.25, 3.12, 1.56, and 0.78 μg/ml. Three plates were prepared for each test organism (n=3 reps/plate×3 pits=9 per compound, per organism). Additional wells were included in duplicate on each plate for color subtraction, ±DMSO, and minus compound controls (n=2 reps/plate×3 pits=6 per control, per organism).

Ten μl of normalized inoculum was added per well. Absorbance at 590 nm was read using a Wallec Victor® Microtiter plate reader; double circular mixing was performed for 10 seconds prior to reading at times 0, 30 and 60 hours. The plates were statically incubated at 28° C. when not being read. Absorbance values were imported into Microsoft Excel® and analyzed graphically.

Utility:

The triaryl cationic compounds described herein, including the pharmaceutically-acceptable salts and isomeric forms thereof, have antibiotic and anti-fungal activity. Consequently, these compounds and pharmaceutical compositions containing the compounds are useful to prevent and to treat pathophysiological conditions due to, caused by, or mediated in whole or in part, by bacterial infection.

Pharmaceutical Compositions:

Another aspect of the invention provides pharmaceutical compositions, for medical use, comprising an active compound, i.e., a Formula I or II compound or a pharmaceutically-acceptable salt thereof, in combination with an acceptable carrier therefor and optionally with other therapeutically-active ingredients or inactive accessory ingredients. The carrier must be pharmaceutically-acceptable in the sense of being compatible with the other ingredients of the formulation and not deleterious to the recipient. The pharmaceutical compositions include those suitable for oral, topical, inhalation, rectal or parenteral (including subcutaneous, intramuscular and intravenous) administration.

The formulations may conveniently be presented in unit dosage form and may be prepared by any of the methods well known in the art of pharmacy. The term “unit dosage” or “unit dose” is denoted to mean a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

Formulations of the present invention suitable for oral administration may be presented as discrete units such as capsules, cachets, tablets, boluses or lozenges, each containing a predetermined amount of the active compound; as a powder or granules; or in liquid form, e.g., as an aqueous solution, suspension, syrup, elixir, emulsion, dispersion, or the like.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine a mixture of the powdered active compound with any suitable carrier.

Formulations suitable for parenteral administration conveniently comprise a sterile preparation of the active compound in, for example, water for injection, saline, a polyethylene glycol solution and the like, which is preferably isotonic with the blood of the recipient.

Useful formulations also comprise concentrated solutions or solids containing the compound of Formula I or II which upon dilution with an appropriate solvent give a solution suitable for parenteral administration.

Preparations for topical or local applications comprise aerosol sprays, lotions, gels, ointments, suppositories etc., and pharmaceutically-acceptable vehicles therefore such as water, saline, lower aliphatic alcohols, polyglycerols such as glycerol, polyethylene glycerol, esters of fatty acids, oils and fats, silicones, and other conventional topical carriers. In topical formulations, the subject compounds are preferably utilized at a concentration of from about 0.1% to 10.0% by weight.

Compositions suitable for rectal administration, comprise a suppository, preferably bullet-shaped, containing the active ingredient and pharmaceutically-acceptable vehicles therefore such as hard fat, hydrogenated cocoglyceride, polyethylene glycol and the like. In suppository formulations, the subject compounds are preferably utilized at concentrations of from about 0.1% to 10% by weight.

Compositions suitable for rectal administration may also comprise a rectal enema unit containing the active ingredient and pharmaceutically-acceptable vehicles therefore such as 50% aqueous ethanol or an aqueous salt solution which is physiologically compatible with the rectum or colon. The rectal enema unit consists of an applicator tip protected by an inert cover, preferably comprised of polyethylene, lubricated with a lubricant such as white petrolatum and preferably protected by a one-way valve to prevent back-flow of the dispensed formula, and of sufficient length, preferably two inches, to be inserted into the colon via the anus. In rectal formulations, the subject compounds are preferably utilized at concentrations of from about 5.0-10% by weight.

Useful formulations also comprise concentrated solutions or solids containing the active ingredient which upon dilution with an appropriate solvent, preferably saline, give a solution suitable for rectal administration. The rectal compositions include aqueous and non-aqueous formulations which may contain conventional adjuvants such as buffers, bacteriostats, sugars, thickening agents and the like. The compositions may be presented in rectal single dose or multi-dose containers, for example, rectal enema units.

Preparations for topical or local surgical applications for treating a wound comprise dressings suitable for wound care. In both topical or local surgical applications, the sterile preparations of compounds of Formula I or II are preferably utilized at concentrations of from about 0. 1% to 5.0% by weight applied to a dressing.

Compositions suitable for administration by inhalation include formulations wherein the active ingredient is a solid or liquid admixed in a micronized powder having a particle size in the range of about 5 μm or less to about 500 μm or liquid formulations in a suitable diluent. These formulations are designed for rapid inhalation through the oral passage from a conventional delivery systems such as inhalers, metered-dose inhalers, nebulizers, and the like. Suitable liquid nasal compositions include conventional nasal sprays, nasal drops and the like, of aqueous solutions of the active ingredient(s).

In addition to the aforementioned ingredients, the formulations of this invention may further include one or more optional accessory ingredient(s) utilized in the art of pharmaceutical formulations, i.e., diluents, buffers, flavoring agents, colorants, binders, surface active agents, thickeners, lubricants, suspending agents, preservatives (including antioxidants) and the like.

The amount of compound of Formula I or II required to be effective for any indicated condition will, of course, vary with the individual subject being treated and is ultimately at the discretion of the medical or veterinary practitioner. The factors to be considered include the condition being treated, the route of administration, the nature of the formulation, the subject's body weight, surface area, age and general condition, and the particular compound to be administered. In general, a suitable effective dose is in the range of about 0.1 to about 500 mg/kg body weight per day, preferably in the range of about 5 to about 350 mg/kg per day, calculated as the non-salt form of Formula I or II. The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

In general, the pharmaceutical compositions of this invention contain from about 0.5 mg to about 1.5 g active ingredient per unit dose and, preferably, from about 7.5 to about 1000 mg per per unit dose. If discrete multiple doses are indicated, treatment might typically be 100 mg of a compound of Formula I or II given from two to four times per day.

The antibacterial and anti-fungal compounds and compositions according to the present invention may be administered prophylactically, chronically, or acutely. For example, such compounds may be administered prophylactically to patients known to be prone to bacterial or fungal infections, or who are known to have been exposed to potentially infectious agents. The subject compounds may also be administered prophylactically to patients suffering other conditions, such as AIDS or other immune-system-suppressing conditions, that render them susceptible to opportunistic infections. In addition to the prevention of such infections, chronic administration of the subject compounds will typically be indicated in treating refractory conditions, such as persistent infection by multiple drug-resistant strains of bacterial or fungus. Acute administration of the subject compounds is indicated to treat, for example, those subjects presenting with classical indications of bacterial or fungal infection.

### EXAMPLES

The following examples are provided for illustrative purposes only. It is understood that the following examples do not limit the invention claimed herein in any way.

BAC Library Construction:

The construction of the soil eDNA BAC library that was used to screen for the production of colored compounds has been described elsewhere (15; co-pending and co-owned U.S. patent application Ser. No. 09/558,712, filed Apr. 26, 2000,). eDNA was isolated directly from soil samples by a freeze-thaw cycle in lysis buffer, followed by extraction with phenol/chloroform and recovery from the aqueous phase by isopropanol precipitation. High molecular weight DNA was purified from the crude DNA samples by two successive pulsed field agarose gel purifications. Size-selected, partially digested high molecular weight DNA was then used to construct a 24,546 member BAC library of soil DNA in the pBeloBAC11 (9) vector. A colored clone from the library, designated P57-G4, was found to produce a dark brown/orange color when grown in LB medium. This discovery prompted a closer examination of the culture broth of this clone.

Characterization of the Acid Precipitate:

The presence of a dark brown color in bacterial cultures is generally characteristic of the production of melanin or a melanin-type polymer that can be collected from culture broth by acid precipitation. Upon acidification of the P57-G4 culture broth, a dark brown precipitate formed, suggesting the presence of melanin in the culture. The dark brown material in the acid precipitate harvested from the P57-G4 (like other bacterial melanin, see references 16, 17) was: 1) insoluble in aqueous acid; 2) soluble in aqueous base; 3) adhered to an anion-exchange column (DEAE-cellulose); and 4) had a molecular weight of >7000 Daltons.

Antibiotic Isolation and Characterization:

The acid precipitate from P57-G4 cultures were examined for additional colored compounds that could be extracted with an organic solvent. Two colored compounds were extracted with methanol from the acid precipitate that formed when the cell-free culture broth of P57-G4 grown in LB for 48 hours (37° C.) was brought to pH 13 with NaOH, stirred for one hour and then acidified to pH 2 with HCl. Using normal phase chromatography, an orange (turbomycin A) and a red (turbomycin B) compound were isolated in approximately a 20:1 ratio from the methanol extract. Both compounds show broad-spectrum antibiotic activity as is demonstrated in Table 2.

TABLE 2
Antibiotic Activities of Synthetic and Natural Turbomycin A and B.

| | | | Turbomycin B | |
|---|---|---|---|---|
| | Turbomycin A | | Syn- | |
| Organisms and Strains | Synthetic | Natural | thetic | Natural |
| Gram Negative | | | | |
| Erwinia herbicola IRQ | +^{a} | + | + | + |
| Escherichia coli HS997 | +/−^{b} | +/− | +/− | +/− |
| Pseudomonas aeruginosa 9020 | −^{c} | − | − | − |
| Salmonella typhimurium LT2 | + | + | + | + |
| Gram Positive | | | | |
| B. cereus var. mycoides 1003 | + | + | + | + |
| Bacillus subtilis BR151pPL | + | + | + | + |
| Enterococcus faecalis 4025 | − | − | − | − |
| Staphylococcus aureus 3001 | + | + | + | + |
| Streptococcus pyogenes 8P01 | + | + | + | + |
| Streptomyces griseus 6501 | + | + | + | + |
| Fungi/Protists | | | | |
| Candida guilliermondii Y001 | +/− | +/− | +/− | +/− |
| Pythium ultimum 1033 | − | − | − | − |
| Fusarium solani (f. sp. | − | − | − | − |
| glycine) 90.1 | | | | |

^{a}(+) 2 to 13 mm zone,
^{b}(+/−) 1 mm zone,
^{c}(−) no zone of inhibition.

In broth culture, the minimum inhibitory concentration (MIC) for synthetic turbomycin A was 6.2 μg/ml for Erwinia herbicola, Bacillus subtilis, Staphylococcus aureus, and Streptococcus pyogenes, and 12.5 μg/ml for Salmonella typhimurium. The natural product and synthetic compound were tested in the same experiment against S. aureus and had the same MIC.

Structure Determination:

HRFABMS data for turbomycin A (Formula I, each R is hydrogen) indicated a molecular formula of C₂₅H₁₈N₃, while 1 - and 2-D NMR experiments suggested the presence of only 5 distinct proton environments and 9 distinct carbon environments. The ortho substituted aromatic ring of the indole was easily identified from the 4-carbon spin system seen in ¹H-¹H RelayH experiments and the nitrogen at position 1 was suggested by the deshielding of C-2, C-7a, and H-2. The remainder of a C-3 substituted indole was confirmed by ¹H-¹³C HMBC correlations from C-3a, -3, -7a and -8 to H-2. All 5 protons and 9 carbons observed by NMR are present in the C-3 substituted indole partial structure.

The structure of turbomycin B (Formula II, each R is hydrogen) was supported by the molecular formula deduced by HRFABMS. However, it was not possible to establish conclusively this structure using spectral data alone. Therefore, synthesis and single crystal X-ray diffraction analysis were used to confirm the structure shown in Formula II.

Synthetic turbomycin B was made by heating indole-3 -carboxyaldehyde and indole in 10% acetic acid, and then oxidizing in situ with tetrachloro-1, 4-benzoquinone to yield an orange compound that was spectroscopically identical to the natural product. The structure of the synthetic material was confirmed by single crystal X-ray diffraction analysis of the perchlorate salt.

For turbomycin B, the presence of the indole was deduced in the same manner as described for turbomycin A and the para substitued phenol was apparent from both the ¹H NMR spectrum and the additional spin system observed in ¹H-¹H RelayH experiments. The structure shown in Formula II was suggested by the molecular formula predicted by HRFABMS, C₂₃H₁₇N₂. As with turbomycin A, the proposed structure of turbomycin B was confirmed by de novo synthesis: Benzaldehyde was heated with indole in 10% acetic acid and the resulting substituted methane was then oxidized in situ with tetrachloro-1,4-benzoquinone to yield a red compound that was spectroscopically identical to the compound isolated from the P57-G4 culture media.

Characterization of the Antibiotic Producing Clone:

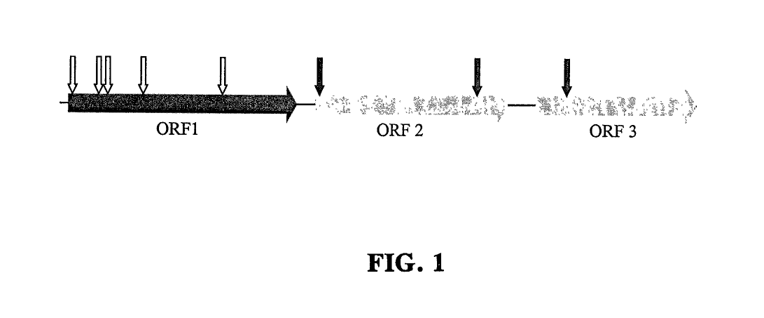

The P57-G4 clone contains a 25-kb insert of eDNA. PstI fragments of P57-G4 were subcloned into pGEM-5Zf+ to generate templates for end sequencing. One of the subclones, G4P8, produced brown pigment when grown in liquid and on solid medium. TLC analysis of supernatant precipitates from P57-G4 and G4P8 cultures revealed that both turbomycin A and B were present in G4P8 cultures, indicating that the genetic information required for color production in P57-G4 cultures was present on the G4P8 subclone. G4P8 was found to contain a 3-kb insert entirely from the P57-G4 environmental insert DNA. Sequence analysis of this insert revealed three ORFs (FIG. 1).

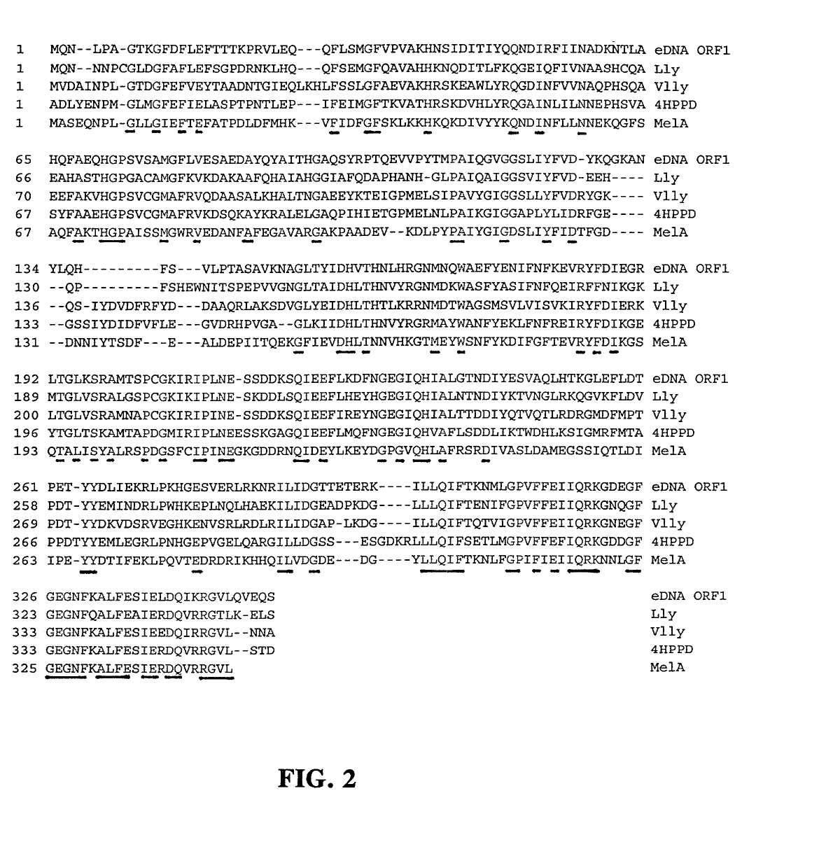

The deduced protein encoded by ORF1 (SEQ. ID. NO. 1) shares extensive similarity with legiolysin (Lly) (SEQ. ID. NO. 2) from Legionella pneumophila (53% identity, 71% similarity), hemolysin (Vlly) (SEQ. ID. NO. 3) from Vibrio vulnificus (54% identity, 71% similarity) (20), 4-hydroxyphenylpyruvate dioxygenase (4HPPD) (SEQ. ID. NO. 4) from Pseudomonas sp. (49% identity, 67% similarity), and MelA (SEQ. ID. NO. 5) from Shewanella colwelliana (45% identity, 63% similarity) (21). FIG. 2 is an amino acid alignment of these sequences. Lly, hemolysin, MelA, and 4HPPD are considered to be members of the 4HPPD family and are believed to be components of the tyrosine/phenylalanine degradation pathway. Identical residues are underlined.

Translation and analysis of the remaining ORFs in G4P8 revealed predicted proteins containing similarity to other putative members of the tyrosine/phenylalanine degradation pathway. The deduced protein product of ORF2 contains weak similarity to an uncharacterized Sinorhizobium meliloti ORF. This ORF is found in the n4 locus of S. meliloti linked to other members of the tyrosine degradation pathway and is believed to be a potential member of that or a related pathway (22).

The deduced protein product of ORF3 contains similarity to a member of the maleylacetoacetate isomerase, which is responsible for the degradation of maleylacotacetate in the tyrosine degradation pathway.

End sequencing of additional PstI clones derived from subcloning of P57-G4 identified the neighboring coding sequence for ORF3 found on G4P8 and an adjacent sequence with homology to homogentisate deoxygenase (hmgA). Homogentisate (HGA) is an intermediate in the tyrosine degradation pathway and is also the polymerization substrate in L. pneumophila cultures. Homologs of hmgA have been identified in only two other non-eukaryotic species, S. meliloti (22) and Streptomyces coelicolor (23). Mutations in hmgA in S. meliloti result in the accumulation of a brown pigmentation, likely homogentisate or an analog (22). Similar observations have been made from cultures of Aspergillus nidulans carrying a mutation in its hmgA gene (24). In L. pneumophila and S. colwelliana, expression of lly and melA, ORF1 homologs, results in the accumulation of HGA-melanin, a dark pigment generated by the spontaneous polymerization of phenolic compounds (25).

Clone G4P8 was mutagenized with transposon GPS-1, and the resulting mutants were screened for color production. The five non-color-producing insertion mutants all carried the transposon within the ORF1 coding sequence (FIG. 1). Of six color-producing insertion mutants, two carried an insertion in the vector, and four carried an insertion in non-vector sequences outside the ORF1 coding sequences. TLC analysis indicated that clones carrying insertions within ORF1 produced neither turbomycin A nor turbomycin B. These results indicate that ORF1 is necessary and sufficient for color production in E. coli.

The methanol extract from the uninoculated LB medium control, treated in the same manner as P57-G4 cultures, did not contain turbomycin A or B. However, the methanol extracts of the acid precipitate harvested from control cultures (E. coli transformed with the BAC vector containing no insert eDNA) contained very small quantities of both turbomycin A and B. The major colored metabolite, turbomycin A, however, was present in approximately 80-fold excess in the culture of P57-G4 compared to the BAC vector control.

The gene encoding legiolysin (lly), which is the gene most closely related to ORF1, was introduced into E. coli. Clones containing lly also produced a melanin-like brown pigment and had elevated levels of triaryl cations in the acid precipitate harvested from the culture broth, strongly suggesting that the formation of these types of triaryl compounds correlates with the presence of HGA-melanin in the culture medium.

Therefore, a cell-free system, containing HGA, was tested for the production of compounds of Formulae I or II. The addition of 1 mM HGA to LB medium resulted in the formation of significant quantities of HGA-melanin pigment by 48 hours. However, the formation of HGA-melanin from HGA (1 μM to 1 mM) in this cell-free system was not sufficient for the production of the orange or red pigments or the compounds of Formulae I or II.

### BIBLIOGRAPHY

1. Britschgi, T. B. & Giovannoni, S. J. (1991). Phylogenic analysis of a natural marine bacterioplankton population by rRNA gene cloning and sequencing. Appl. Environ. Microbiol. 57, 1707-1713.

2. Suau, A., Bonnet, R., Sutren, M., Godon, J. J., Gibson, G. R., Bonnet, R., Malene, S. & Dore, J. (1999). Direct analysis of genes encoding 16S rRNA from complex communities reveals many novel molecular species within the human gut. Appl. Environ. Microbiol. 65, 4799-4807.

3. Kudo, T., Ohkuma, N., Moriya, S., Noda, S. & Ohtoko, K. (1999). Molecular and phylogenetic identification of the intestinal anaerobic microbial community in the hindgut of the termite, Reticulitermes spermatus, without cultivation. Extremophiles 2, 151 - 161.

4. Ward, D. M., Weller, R. & Bateson, M. M. (1990). 16S rRNA sequences reveal numerous uncultured microorganisms in a natural community. Nature 345, 63-65.

5. Bintrim, S. B., Donohue, T. J., Handelsman, J., Roberts, G. P. & Goodman, R. M. (1997). Molecular phylogeny of Archaea from soil. Proc. Natl. Acad. Sci. U.S.A. 94, 277-282.

6. Borneman, J., Skroch, P. W., O Sullivan, K. M., Palus, J. A., Rumjanek, N. G., Jansen, J. L., Nienhuis, J. & Triplett, E. W. (1996). Molecular microbial diversity of an agricultural soil in Wisconsin. Appl. Environ. Micro biol. 62, 1935-1943.

7. Stackebrandt, B., Liesack, W. & Goebel, B. M. (1993). Bacterial diversity in a soil sample from a subtropical Australian environment as determined by 16S rDNA analysis. FASEB J. 7, 232-236.

8. Handelsman, J., Rondon, M. R., Brady, S. F., Clardy, J. & Goodman, R. M. (1998). Molecular biological access to the chemistry of unknown soil microbes: a new frontier for natural products. Chem. Biol. 5, R245-R249.

9. Shizuya, H., Birren, B., Kim, U. J., Mancino, V., Slepak, Y., Tachiri, Y. & Simon, M.(1992). Cloning and stable maintenance of 300-kilobase-pair fragments of human DNA in Escherichia coli using an F-factor-based vector. Proc. Natl. A cad. Sci. U.S.A. 89, 8794-8797.

10. Torsvik, V., Goksoyr, J. & Daae, F. L. (1990). High diversity of DNA of soil bacteria. Appl. Environ. Microbiol. 56, 782-787.

11. Torsvik, V., Salte, K., Sorheim, R. & Goksoyr, J. (1990). Comparison of phenotypic diversity and heterogeneity in a population of soil bacteria. Appl. Environ. Microbiol. 56, 776-781.

12. Liesack, W. & Stackebrandt, E. (1992). Occurrence of novel groups of the domain Bacteria as revealed by analysis of genetic material isolated from an Australian terrestrial environment. J. Bacteriol. 174, 5072-5078.

13. Hugenholtz, P., Goebel B. M. & Pace, N. R. (1998). Impact of culture-independent studies on the emerging phylogenetic view of bacterial diversity. J. Bacteriol. 180, 4765- 4774.

14. Zhou, J., Bruns, M. A. & Tiedje, J. M. (1996). DNA recovery from soils of diverse composition. Appl. Environ. Microbiol. 62, 316-322.

15. Rondon, M. R., August, P. R., Betterman, A. D., Brady, S. F., Grossman, T. H., Liles, M. R., Loiacono, K. A., Lynch, B. A., MacNeil, 1.A., Charles, M., Tiong, C. L., Gilman, M., Osburne, M. S., Clardy, J., Hendelsman, J. & Goodman, R. M. (2000). Cloning the soil metagenome: a strategy for accessing the genetic and functional diversity of uncultured microorganisms. Appl. Environ. Microbiol., 66 in press.

16. Gibson, L. F. & George, A. M. (1998). Melanin and novel precursors from Aeromonas media. FEMS Microbiol. Lett. 169, 26 1-268. (and references therein).

17. Schmidt, R., Krein, R. & Regnier, M. (1996). The use of diethylaminoethylcellulose membrane filters in a bioassay to quantify melanin synthesis. Anal. Biochem. 235, 113-118.

18. Budzikiwicz, H., Eckau, H. & Ehrenberg, M. (1972). Bis (3-indolyl-)-3H-indolyliden-methan, em von Saccharomyces Cerevisiae Gebildeter Farbstoff Tetrahedron Lett. 36, 3807-38 10.

19. Stupnikova, T. V., Rybenko, L. A., Skorobogatova, Z. M. & Sheinkman, A. K. (1978). Tris(3-indoyl)methyl perchlorates as new dyes of the triarylmethane series. Khim. Geterotsikl. Soedin. 3, 416.

20. Chang, T. M., Chuang, Y. C., Su, J. H. & Chang, M. C. (1997). Cloning and sequence analysis of a novel hemolysin gene (vllY) from Vibrio vulnificus. Appl. Environ. Microbiol. 63, 385 1-3857.

21. Fuqua, W. C., Coyne, V. E., Stein, D. C., Lin, C. M. & Weiner, R. M. (1991). Characterization of melA: a gene encoding melanin biosynthesis from the marine bacterium Shewanella colwelliana. Gene, 109, 131-136.

22. Milcamps, A & de Bruijn, F. J. (1999). Identification of a novel nutrient-deprivationinduced Sinorhizobium meliloti gene (hmgA) involved in the degradation of tyrosine. Microbiology 145, 935-947.

23. Redenbach, M., Kieser, H. M., Denapaite, D., Eichner, A., Cullum, J., Kinashi, H. & Hopwood, D.A. (1996). A set of ordered cosmids and a detailed genetic and physical map for the 8 Mb Streptomyces coelicolor A3(2) chromosome. Mol. Microbiol. 21, 77-96.

24. Fernandez-Canon, J. M. & Penalva, M. A. (1995). Molecular characterization of a gene encoding a homogentisate dioxygenase from Aspergillus nidulans and identification of its human and plant homologues. J. Biol. Chem. 270,21199-21205.

25. Wintermeyer, E., Flugel, M., Ott, M., Steinert, M., Rdest, U., Mann, K. H. & Hacker, J. (1994). Sequence determination and mutational analysis of the lly locus of Legionella pneumophila. Infect. Immun. 62, 1109-1117.

26. Sambrook, J., Fritsch, E. F. & Maniatis, T. (1989). Molecular Cloning: A Laboratory Manural. (2nd ed.), Cold Spring Harbor Laboratory Press, Cold Spring Harbor, N.Y.

27. Altschul, S. F., Gish, W., Miller, W., Myers, E. W. & Lipman, D. J. (1990). Basic local alignment search tool. J. Mol. Biol. 215, 403-410.

28. Saiki, R. K., Gelfand, D. H., Stoffel, S., Scharf, S. J., Higuchi, R., Horn, G. T., Mullis, K. B. & Erlich, H. A. (1988). Primer-directed enzymatic amplification of DNA with a thermostable DNA polymerase. Science 239, 487-491.

29. Dhingra, O. D. & Sinclair, J. B. (1985). Basic plant pathology methods. Boca Raton, Fla.: CRC Press, Inc., p 308.

What is claimed is:

1. A pharmaceutical composition for treating microbial or fungal infections comprising an antibiotic-effective or an antifungal-effective amount of a compound selected from the group consisting of: 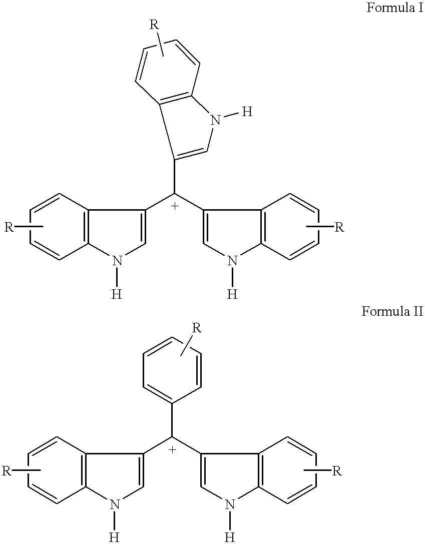
wherein each R is independently selected from the group consisting of hydrogen, halo, hydroxy, amino, unsubstituted C₁ to C₆-alkyl, and C₁ to C₆-alkyl substituted with one or more of halo, hydroxy, or amino; and pharmaceutically-acceptable salts thereof; in combination with a pharmaceutically-acceptable carrier.

2. The pharmaceutical composition of claim 1, wherein the compound has a structure as shown in Formula I.

3. The pharmaceutical composition of claim 2, wherein each R is independently selected from the group consisting of hydrogen, halo, hydroxy, and unsubstituted C₁ to C₆-alkyl.

4. The pharmaceutical composition of claim 2, wherein each R is hydrogen.

5. The pharmaceutical composition of claim 1, wherein the compound has a structure as shown in Formula II.

6. The pharmaceutical composition of claim 5, wherein each R is independently selected from the group consisting of hydrogen, halo, hydroxy, and unsubstituted C₁ to C₆-alkyl.

7. The pharmaceutical composition of claim 5, wherein each R is hydrogen.

8. A method of treating bacterial or fungal infection in a subject in need thereof, the method comprising administering to the subject an antibiotic- or antifungal-effective amount of a compound selected from the group consisting of: 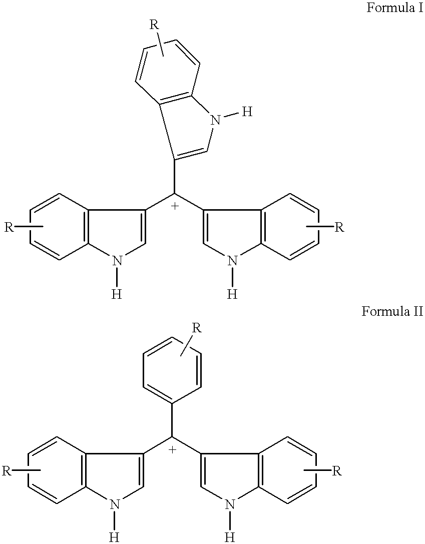
wherein each R is independently selected from the group consisting of hydrogen, halo, hydroxy, amino, unsubstituted C₁ to C₆-alkyl, and C₁ to C₆-alkyl substituted with one or more of halo, hydroxy, or amino; and pharmaceutically-acceptable salts thereof; in combination with a pharmaceutically-acceptable carrier.

9. The method of claim 8, wherein a compound as shown in Formula I is administered to the subject.

10. The method of claim 9, wherein a compound wherein each R is independently selected from the group consisting of hydrogen, halo, hydroxy, and unsubstituted C₁ to C₆-alkyl is administered to the subject.

11. The method of claim 9, wherein a compound wherein each R is hydrogen is administered to the subject.

12. The method of claim 9, wherein the compound is administered to a human subject.

13. The method of claim 9, wherein the bacterial or fungal infection treated is selected from the group consisting of Erwinia herbicola, Escherichia coli, Salmonella typhimurium, Bacillus cereus, Bacillus subtilis, Staphylococcus aureus, Streptococcus pyogenes, Streptomyces griseus, and Candida guilliermondii.

14. The method of claim 8, wherein a compound as shown in Formula II is administered to the subject.

15. The method of claim 14, wherein a compound wherein each R is independently selected from the group consisting of hydrogen, halo, hydroxy, and unsubstituted C₁ to C₆-alkyl is administered to the subject.

16. The method of claim 14, wherein a compound wherein each R is hydrogen is administered to the subject.

17. The method of claim 14, wherein the compound is administered to a human subject.

18. The method of claim 14, wherein the bacterial or fungal infection treated is selected from the group consisting of Erwinia herbicola, Escherichia coli, Salmonella typhimurium, Bacillus cereus, Bacillus subtilis, Staphylococcus aureus, Streptococcus pyogenes, Streptomyces griseus, and Candida guilliermondii.

19. A compound selected from the group consisting of: 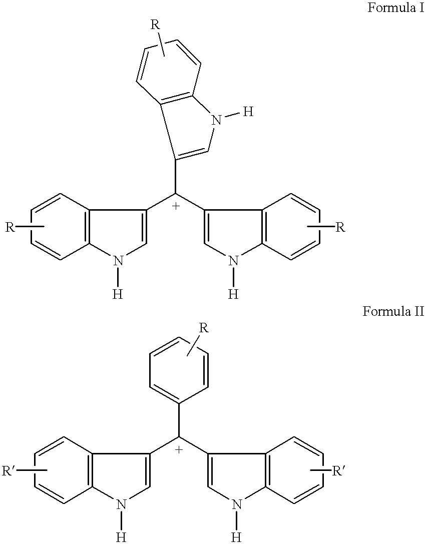
wherein each R is independently selected from the group consisting of hydrogen, halo, hydroxy, amino, unsubstituted C₁ to C₆-alkyl, and C₁ to C₆-alkyl substituted with one or more of halo, hydroxy, or amino, provided that each R group is not simultaneously hydrogen;
each R′ is independently selected from the group consisting of hydrogen, halo, hydroxy, amino, unsubstituted C₁ to C₆-alkyl, and C₁ to C₆-alkyl substituted with one or more of halo, hydroxy, or amino;
and salts thereof.

20. The compound of claim 19, wherein the compound has a structure as shown in Formula I.

21. The compound of claim 20, wherein each R is independently selected from the group consisting of hydrogen, halo, hydroxy, and unsubstituted C₁ to C₆-alkyl, provided that each R is not simultaneously hydrogen.

22. The compound of claim 19, wherein the compound has a structure as shown in Formula II.

23. The compound of claim 22, wherein each R′ is independently selected from the group consisting of hydrogen, halo, hydroxy, and unsubstituted C₁ to C₆-alkyl.

24. The compound of claim 22, wherein each R′ is hydrogen.

25. An isolated polypeptide as shown in SEQ. ID. NO. 1.

26. An isolated polynucleotide encoding a polypeptide as shown in SEQ. ID. NO. 1.
